SEQUENCE LISTING (18 sequences)

SEQ ID NO: 1 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 2 - protein, 531 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPHHHHHHDYKDDDDKI

SEQ ID NO: 3 - protein, 340 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAHHHHHHDYKDDDDKI

SEQ ID NO: 4 - DNA, 1878 bp, Homo sapiens
atgatattcctcacggcactgcctctgttctggattatgatttcagcctcccgagggggtcactggggtgcctggatgccctcgtccatctcggccttcgaaggcacgtgcgtctccatcccctgccgctttgacttcccggatgagctgcggcccgctgtggtgcatggtgtctggtacttcaatagcccctaccccaagaactaccccccggtggtcttcaagtcgcgcacccaagtagtccacgagagcttccagggccgcagccgcctcctgggggacctgggcctgcgaaactgcaccctcctgctcagcaacgtcagccccgagctgggcgggaagtactacttccgtggggacctgggcggctacaaccagtacaccttctcagagcacagcgtcctggatatcgtcaacacccccaacatcgtggtgcccccagaggtggtggcaggcacggaagtggaggtcagctgcatggtgccggacaactgcccagagctgcgccctgagctgagctggctgggccacgaggggctgggggagcccgctgtgctgggccggctgcgggaggacgagggcacctgggtgcaggtgtcactgctgcacttcgtgcccacgagggaggccaacggccacaggctgggctgccaggcctccttccccaacaccaccctgcagttcgagggctacgccagcatggacgtcaagtaccccccggtgattgtggagatgaactcctcggtggaggccatcgagggctcccacgtgagcctgctctgtggggctgacagcaaccccccgccgctgctgacctggatgcgggacgggacagtcctccgggaggcggtggccgagagcctgctcctggagctggaggaggtgacccccgccgaagacggcgtctatgcctgcctggccgagaatgcctatggccaggacaaccgcaccgtggggctcagtgtcatgtatgcaccctggaagccaacagtgaacgggacaatggtggccgtagagggggagacggtctctatcttgtgctccacacagagcaacccggaccctattctcaccatcttcaaggagaagcagatcctgtccacggtcatctacgagagcgagctgcagctggagctgccggccgtgtcacccgaggatgatggagagtactggtgtgtggctgagaaccagtatggccagagggccaccgccttcaacctgtctgtggagttcgcccctgtgctcctcctggagtcccactgcgcggcagcccgagacacggtgcagtgcctgtgcgtggtgaagtccaacccggagccgtccgtggcctttgagctgccatcgcgcaatgtgaccgtgaacgagagcgagcgggagttcgtgtactcggagcgcagcggcctcgtgctcaccagcatcctcacgctgcgggggcaggcccaggccccgccccgcgtcatctgcaccgcgaggaacctctatggcgccaagagcctggagctgcccttccagggagcccatcgactgatgtgggccaagatcgggcctgtgggcgccgtggtcgcctttgccatcctgattgccatcgtctgctacattacccagacacgcaggaaaaagaacgtgacagagagccccagcttctcggcaggggacaaccctcccgtcctgttcagcagcgacttccgcatctctggggcaccagagaagtacgagagcgagaggcgcctgggatctgagaggaggctgctgggccttcggggtgagcccccagagctggacctgagctattctcactcggacctggggaaacggcccaccaaggacagctacacgctgacggaggagctagctgagtatgctgaaatccgggtcaag

SEQ ID NO: 5 - DNA, 1593 bp, Homo sapiens
atgatattcctcacggcactgcctctgttctggattatgatttcagcctcccgagggggtcactggggtgcctggatgccctcgtccatctcggccttcgaaggcacgtgcgtctccatcccctgccgctttgacttcccggatgagctgcggcccgctgtggtgcatggtgtctggtacttcaatagcccctaccccaagaactaccccccggtggtcttcaagtcgcgcacccaagtagtccacgagagcttccagggccgcagccgcctcctgggggacctgggcctgcgaaactgcaccctcctgctcagcaacgtcagccccgagctgggcgggaagtactacttccgtggggacctgggcggctacaaccagtacaccttctcagagcacagcgtcctggatatcgtcaacacccccaacatcgtggtgcccccagaggtggtggcaggcacggaagtggaggtcagctgcatggtgccggacaactgcccagagctgcgccctgagctgagctggctgggccacgaggggctgggggagcccgctgtgctgggccggctgcgggaggacgagggcacctgggtgcaggtgtcactgctgcacttcgtgcccacgagggaggccaacggccacaggctgggctgccaggcctccttccccaacaccaccctgcagttcgagggctacgccagcatggacgtcaagtaccccccggtgattgtggagatgaactcctcggtggaggccatcgagggctcccacgtgagcctgctctgtggggctgacagcaaccccccgccgctgctgacctggatgcgggacgggacagtcctccgggaggcggtggccgagagcctgctcctggagctggaggaggtgacccccgccgaagacggcgtctatgcctgcctggccgagaatgcctatggccaggacaaccgcaccgtggggctcagtgtcatgtatgcaccctggaagccaacagtgaacgggacaatggtggccgtagagggggagacggtctctatcttgtgctccacacagagcaacccggaccctattctcaccatcttcaaggagaagcagatcctgtccacggtcatctacgagagcgagctgcagctggagctgccggccgtgtcacccgaggatgatggagagtactggtgtgtggctgagaaccagtatggccagagggccaccgccttcaacctgtctgtggagttcgcccctgtgctcctcctggagtcccactgcgcggcagcccgagacacggtgcagtgcctgtgcgtggtgaagtccaacccggagccgtccgtggcctttgagctgccatcgcgcaatgtgaccgtgaacgagagcgagcgggagttcgtgtactcggagcgcagcggcctcgtgctcaccagcatcctcacgctgcgggggcaggcccaggccccgccccgcgtcatctgcaccgcgaggaacctctatggcgccaagagcctggagctgcccttccagggagcccatcgactgatgtgggccaagatcgggcctcatcaccatcatcaccatgattacaaggatgacgacgataagatc

SEQ ID NO: 6 - DNA, 1020 bp, Homo sapiens
atgatattcctcacggcactgcctctgttctggattatgatttcagcctcccgagggggtcactggggtgcctggatgccctcgtccatctcggccttcgaaggcacgtgcgtctccatcccctgccgctttgacttcccggatgagctgcggcccgctgtggtgcatggtgtctggtacttcaatagcccctaccccaagaactaccccccggtggtcttcaagtcgcgcacccaagtagtccacgagagcttccagggccgcagccgcctcctgggggacctgggcctgcgaaactgcaccctcctgctcagcaacgtcagccccgagctgggcgggaagtactacttccgtggggacctgggcggctacaaccagtacaccttctcagagcacagcgtcctggatatcgtcaacacccccaacatcgtggtgcccccagaggtggtggcaggcacggaagtggaggtcagctgcatggtgccggacaactgcccagagctgcgccctgagctgagctggctgggccacgaggggctgggggagcccgctgtgctgggccggctgcgggaggacgagggcacctgggtgcaggtgtcactgctgcacttcgtgcccacgagggaggccaacggccacaggctgggctgccaggcctccttccccaacaccaccctgcagttcgagggctacgccagcatggacgtcaagtaccccccggtgattgtggagatgaactcctcggtggaggccatcgagggctcccacgtgagcctgctctgtggggctgacagcaaccccccgccgctgctgacctggatgcgggacgggacagtcctccgggaggcggtggccgagagcctgctcctggagctggaggaggtgacccccgccgaagacggcgtctatgcctgcctggccgagaatgcctatggccaggacaaccgcaccgtggggctcagtgtcatgtatgcacatcaccatcatcaccatgattacaaggatgacgacgataagatc

SEQ ID NO: 7 - DNA, 40 bp, Artificial
  SYNTHETIC CONSTRUCT
gatcgatcagatctgccgccatgatattcctcacggcact

SEQ ID NO: 8 - DNA, 45 bp, Artificial
  SYNTHETIC CONSTRUCT
tagtactagaattctcatcacttgacccggatttcagcatactca

SEQ ID NO: 9 - DNA, 40 bp, Artificial
  SYNTHETIC CONSTRUCT
gatcgatctctagagccgccatgatattcctcacggcact

SEQ ID NO: 10 - DNA, 86 bp, Artificial
  SYNTHETIC CONSTRUCT
tagtactagaattctcatcagatcttatcgtcgtcatccttgtaatcatggtgatgatggtgatgaggcccgatcttggcccacat

SEQ ID NO: 11 - DNA, 40 bp, Artificial; identical to SEQ ID NO: 9
  SYNTHETIC CONSTRUCT

SEQ ID NO: 12 - DNA, 86 bp, Artificial
  SYNTHETIC CONSTRUCT
tagtactagaattctcatcagatcttatcgtcgtcatccttgtaatcatggtgatgatggtgatgtgcatacatgacactgagccc

SEQ ID NO: 13 - DNA, 27 bp, Artificial
  SYNTHETIC CONSTRUCT
tcagcgatcactcactcgctgtacaga

SEQ ID NO: 14 - DNA, 51 bp, Artificial
  ARTIFICIAL CONSTRUCT
aggcccgatcttggcccacatcagtcgtgcatacatgacactgagccccac

SEQ ID NO: 15 - DNA, 46 bp, Artificial
  SYNTHETIC CONSTRUCT
ggggctcagtgtcatgtatgcacgactgatgtgggccaagatcggg

SEQ ID NO: 16 - DNA, 31 bp, Artificial
  SYNTHETIC CONSTRUCT
gggtcagccacgcaggctgcccccagctcct

SEQ ID NO: 17 - DNA, 40 bp, Artificial; identical to SEQ ID NO: 7
  SYNTHETIC CONSTRUCT

SEQ ID NO: 18 - DNA, 44 bp, Artificial
  SYNTHETIC CONSTRUCT
gatcgatcgaattctcatcacttgacccggatttcagcatactc

(19) United States
(12) Patent Application Publication
(10) Pub. No.: US 2006/0088912 A1
(43) Pub. Date: Apr. 27, 2006

(54) Compositions and methods of purifying myelin-associated glycoprotein (MAG)

(75) Inventors: Gouying Yan, Lexington, MA (US); Yuhong Xie, Chestnut Hill, MA (US); Janet E. Paulsen, Londonderry, NH (US); Jimin Zhang, Chestnut Hill, MA (US); Dionna Rookey, Philadelphia, PA (US); Brian Bates, Chelmsford, MA (US); Zhijian Lu, Bedford, MA (US); Robert Mark, Lawrenceville, NJ (US); Susie J. Campos, Hackettstown, NJ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: WYETH, Madison, NJ (US)

(21) Appl. No.: 11/180,855

(22) Filed: Jul. 13, 2005

Related U.S. Application Data
(60) Provisional application No. 60/587,893, filed on Jul. 14, 2004.
(60) Provisional application No. 60/588,239, filed on Jul. 15, 2004.

(51) Int. Cl. C12P 21/06; C07H 21/04; C12N 5/06; C07K 14/47

(52) U.S. Cl. 435/69.1; 435/455; 435/358; 530/395; 536/23.5

(57) ABSTRACT

The present invention provides compositions and methods useful for purifying recombinant myelin-associated glycoprotein (MAG) and fragments thereof. In particular, the invention provides a one-step purification method for MAG and MAG fragments. Novel forms of human recombinant MAG protein are also disclosed in addition to methods of reliably producing and storing stable recombinant MAG proteins.

### RELATED APPLICATIONS

This application claims priority from U.S. provisional application 60/587,893 filed Jul. 14, 2004, and U.S. provisional application 60/588,239 filed Jul. 15, 2004, the subject matter of which are incorporated herein by reference.

### FIELD OF THE INVENTION

The present invention relates to a membrane-bound cell adhesion molecule belonging to the superfamily of IgG-genes. In particular, the invention pertains to myelin-associated glycoprotein (MAG) and methods of MAG protein recovery and purification.

### BACKGROUND OF THE INVENTION

Pathological or traumatic damage to central nervous system (CNS) nerve fibers results in permanent loss of function in adult mammals. This lack of nerve regeneration is attributable in part to inhibitory factors found in myelin. Myelin-associated glycoprotein (MAG) is an abundant myelin protein that inhibits neurite outgrowth, which makes its role in regeneration a possible target for the development of therapeutics to promote recovery following human CNS injury.

MAG is a 100-kDa glycoprotein with five extracellular Ig-like domains, a single transmembrane domain and a cytoplasmic domain that occurs in two developmentally regulated forms that differ only in the cytoplasmic domains due to alternative mRNA splicing. Axonal regeneration in the adult CNS has been shown to be inhibited by proteins in myelin, including MAG and NOGO. While the NOGO receptor (NgR) had been identified as an axonal GPI-anchored protein, the MAG receptor remains elusive.

Recently, MAG was shown to bind directly, with high affinity, to NgR and to inhibit axonal regeneration through interaction with NgR (Domeniconi, M. et al. Myelin-associated glycoprotein interacts with the Nogo66 receptor to inhibit neurite outgrowth. Neuron 35: 283-290, 2002; Liu, B. P. et al. Myelin-associated glycoprotein as a functional ligand for the Nogo-66 receptor. Science 297: 1190-1193, 2002). Experiments blocking NgR from interacting with MAG prevented inhibition of neurite outgrowth by MAG (Domeniconi, M. et al. Neuron 35: 283-290, 2002). This interaction indicates that MAG and Nogo-66 activate NgR independently and serve as redundant NgR ligands that may limit axonal regeneration after CNS injury.

However, currently used purification techniques are not sufficient for purification of a MAG protein to the level of purity and with the consistency desired for either a human therapeutic product or a reliable research tool. Many methods for producing recombinant MAG involve the use of fusion proteins. MAG fusion proteins, such as MAG-GST fusion proteins, are only very weakly expressed and are very unstable (Kursula P. (2000, Ph.D. Thesis). Cytoplasmic domains of the Myelin-Associated Glycoprotein. Acta Universitatis Ouluensis: D Medica 594. University of Oulu, Finland). Methods of purifying MAG-Fc, recombinant extracellular domains of MAG fused to the Fc fragment of human immunoglobulin, from different neuroblastoma cells, isolated neurons and whole brain or spinal cord are also known in the art (See, for example, Kelm et al., Current Biol., 4, pp. 965-72 (1994)). However, prior to performing assays or producing anti-MAG antibodies, the Fc fusion must be enzymatically cleaved resulting in an impure, unstable cleaved MAG protein. Thus, scientists have to use a combination of traditional chromatographic techniques to purify the desired cleaved MAG. Frequently, even high resolution affinity chromatography steps may not afford sufficient resolution of the desired Fc-cleaved MAG from other components due to common sites of interaction.

Accordingly, there exists a need in the art for methods of efficient purification of MAG without the use of an Fc fusion. There is also a need for improved compositions and methods for nerve regeneration.

### SUMMARY OF THE INVENTION

The present invention provides compositions and methods useful for purifying recombinant myelin-associated glycoprotein (MAG) and fragments thereof. In particular, the invention provides a one-step purification method for MAG and MAG fragments. Novel forms of human recombinant MAG protein are also disclosed in addition to methods of reliably producing and storing stable recombinant MAG proteins.

The invention is based in part on the discovery that a single step purification method can reliably provide highly purified MAG or fragments thereof. As shown in the Examples, MAG and fragments thereof can be purified to greater than 96% purity as confirmed by size exclusion chromatography (SEC). The functionality of the purified protein was confirmed through an inhibition of neurite outgrowth assay which showed that MAG purified according to the methods of this invention inhibits neurite growth at levels comparable to a commercially available MAG-Fc protein.

In one aspect, the present invention discloses a method of purifying recombinant extracellular domain myelin associated glycoprotein (MAG) constructs comprising the steps of: transfecting cells with a vector having a nucleic acid sequence encoding an affinity-tagged MAG construct and capable of expressing the affinity-tagged MAG construct comprising at least one Ig domain; culturing the transfected cells in a medium such that the cells express the affinity-tagged MAG construct; contacting a MAG construct-containing medium with a metal ion affinity chromatography resin, charged with a divalent metal ion; and eluting a purified affinity-tagged MAG construct. The divalent metal ion used in the metal affinity resin can be nickel, cobalt, copper, cadmium, calcium, iron, zinc, or strontium. In certain embodiments, the divalent metal ion can be nickel or cobalt. Preferably, the resin can be nickel-nitrilotriacetic acid (Ni—NTA) resin or TALON™ resin. The cells are cultured to confluency in a medium comprising FBS and then changed to a serum-free medium. Methotrexate is used in the culture medium.

In another aspect, the invention discloses expressing affinity-tagged MAG with a polyhistidine tail and/or a FLAG tag with an amino acid sequence DYKDDDDK. The method further includes expressing affinity-tagged MAG comprising at least two Ig domains, at least three Ig domains, at least four Ig domains, or at least five Ig domains. Culturing the transfected cells results in the expression of glycosylated MAG, wherein the glycosylation is substantially identical to that of human endogenous MAG. In certain embodiments, Chinese Hamster Ovary (CHO) cells can be stably transfected with a vector encoding MAG or a MAG fragment.

In another aspect, the invention discloses eluting the purified affinity-tagged MAG by changing the pH, or adding a chelating agent (e.g., EDTA and EGTA) and/or a competitive ligand (e.g., imidazole, histamine, glycine, and ammonium chloride). The chelating agent can be ethylenediamine tetraacetic acid (EDTA) in an eluting solution having a pH greater than about 7. The competitive ligand can be imidazole.

In another aspect, the method provides storage conditions that retain the stability of the purified MAG. The purified affinity-tagged MAG can be stored in a buffer comprising Na₂HPO₄, NaCl, and a pH greater than about 7.0, in a buffer comprising imidazole, or in a buffer comprising a detergent. In some embodiments, the detergent can be a nonionic detergent. Examples of nonionic detergents useful in the present invention include, but are not limited to, octoxynol-9 (TRITON X-100, Rohm & Haas), polysorbate 80 (TWEEN 80, ICI Americas, Inc., Wilmington Del.), polysorbate 20 (TWEEN 20, ICI Americas, Inc.) and laureth-4 (BRIJ 30, ICI Americas, Inc.). In certain embodiments, the detergent is Tween 20.

Another aspect of the invention discloses purified, glycosylated human recombinant extracellular domain myelin associated glycoprotein (MAG) constructs prepared by the methods disclosed herein, wherein the eluted purified affinity-tagged MAG construct is greater than about 90% pure, or preferably greater than about 95% pure. The MAG construct can have an amino acid sequence substantially homologous to the amino acid sequence depicted in SEQ ID NO:1. The MAG construct can have an amino acid sequence that is substantially homologous with the amino acid sequence depicted in SEQ ID NO:2. The MAG construct can have an amino acid sequence that is substantially homologous with the amino acid sequence depicted in SEQ ID NO: 3.

In yet another aspect, the invention discloses a method for producing an extracellular domain myelin associated glycoprotein (MAG) comprising: contacting a MAG-containing media with an immobilized metal affinity chromatography (IMAC) resin charged with a divalent metal ion; washing the IMAC resin with at least one IMAC wash solution; and eluting the IMAC resin with an eluting solution to obtain a purified MAG solution. The method further includes selecting a divalent metal ion from the group consisting of nickel, cobalt, copper, iron, calcium and zinc. In certain embodiments, the divalent metal ion is nickel or cobalt. The method further comprises culturing transfected cells to confluence in medium comprising about 10% FBS and about 100 nM methotrexate, such that the cells express a MAG construct comprising at least one Ig domain.

In another aspect of the invention, methods of storing MAG to prevent protein destabilization and precipitation are disclosed. For example, a MAG fragment, MAG(1-3) which comprises SEQ ID 3 and has three Ig domains, is stable at both room temperature at 4° C. for at least 12 weeks in sodium phosphate buffer (Na₂HPO₄), pH 7.2 in both high (500 mM) and low (150 mM) salt conditions. Neither the metal affinity resin nor the salt concentration have any effect on the purity or stability of MAG(1-3). However, buffer containing imidazole is slightly better at retaining the stability of MAG(1-5) compared to sodium phosphate buffer with or without detergent. Temperature also affects the stability of MAG(1-5) (SEQ ID 2). At room temperature, aggregation of purified MAG(1-5) increased from 3 to 10% over twelve weeks.

Recombinant MAG protein and fragments thereof of the present invention can be used as immunogens or selection targets in generating MAG-specific antibodies. In addition, the recombinant MAG protein and fragments thereof can be used in assays for studying NOGO receptor interactions with its ligands as well as in development of therapeutic agents blocking interactions for treatment of spinal cord injuries, and cerebral ischemic injuries.

Another aspect of the invention provides molecules that specifically bind to purified MAG or fragments thereof. The binding molecule may be an antibody, antibody fragment, or other molecule. The invention also provides methods for producing a binding molecule that specifically recognizes MAG or fragments thereof.

Other features and advantages of the invention will become apparent to one of skill in the art from the following detailed description, the drawings, and from the claims.

### BRIEF DESCRIPTION OF THE FIGURES

FIG. 1 is a schematic illustration of various MAG fragments of the present invention;

FIG. 2A is a purification chromatogram of MAG(1-5) purified using a TALON™ affinity column;

FIG. 2B is a purification chromatogram of MAG(1-5) purified using a nickel affinity column;

FIG. 3 is an SDS PAGE showing purified MAG(1-5) and MAG(1-3) following TALON™ or Ni—NTA column purification;

FIG. 4 is bar graph demonstrating the inhibition of neurite outgrowth of rat cerebellar granular neurons treated with MAG(1-5) purified using methods of the present invention;

FIG. 5A is bar graph of UV absorption demonstrating the stability of MAG( 1-3) following three cycles of freeze/thaw;

FIG. 5B is an purification chromatogram from size exclusion chromatography (SEC) of MAG(1-3) demonstrating that there is no protein destabilization or aggregation following three cycles of freeze/thaw;

FIG. 6A is bar graph of UV absorption demonstrating the stability of MAG1-5 following three cycles of freeze/thaw;

FIG. 6B is a purification chromatogram from size exclusion chromatography (SEC) of MAG1-5 demonstrating that there is no protein destabilization or aggregation following three cycles of freeze/thaw;

FIG. 7 is graph of percent purity of MAG1-3 by SEC at various time points at following storage at room temperature (RT) or 4° C.;

FIG. 8A is graph of percent purity of MAG1-5 by SEC at various time points at following storage at room temperature (RT) or 4° C.;

FIG. 8B is purification chromatogram from size exclusion chromatography (SEC) of MAG1-5 demonstrating that aggregation increased following 12 weeks of storage at room temperature (RT).

### DETAILED DESCRIPTION OF THE INVENTION

The practice of the present invention employs, unless otherwise indicated, conventional methods of analytical biochemistry, microbiology, molecular biology and recombinant DNA techniques within the skill of the art. Such techniques are explained fully in the literature.

The terminology used herein is for describing particular embodiments and is not intended to be limiting. Unless defined otherwise, all scientific and technical terms are to be understood as having the same meaning as commonly used in the art to which they pertain. For the purposes of the present invention, the following terms are defined below:

The term “MAG,” as used herein, refers to a member of the immunoglobulin (IG) superfamily containing five extracellular Ig-like domains, which is substantially homologous and functionally equivalent to proteins comprising SEQ ID NO:1 (GenBank Accession No. P20916) or peptides comprising SEQ ID NO: 1 with conservative amino acid or non-amino acid substitutions, or functional truncations or addition fragments thereof as described below. Non-limiting examples of mammalian MAG sequences include, but are not limited to, human isoform (GenBank Accession Nos. AAH53347); rat (Rattus norvegicus) (GenBank Accession Nos. NP_{—}058886, BNRT3 and BNRT3S); and mouse (Mus musculus) (GenBank Accession Nos. NP_{—}034888, AAA39487). MAG is intended to cover MAG with conservative amino acid substitutions that result in functional and non-functional MAG as demonstrated by the present invention. MAG is a I 00-kDa glycoprotein with five extracellular Ig-like domains, a single transmembrane domain and a cytoplasmic domain that occurs in two developmentally regulated forms that differ only in the cytoplasmic domains due to alternative mRNA splicing. The extracellular domain of MAG has eight sites for N-linked glycosylation and contains about 30% by weight carbohydrate. The oligosaccharides are very heterogeneous. In addition, MAG is a sialic acid-binding protein and its first four Ig-like domains are homologous to those of other sialic acid binding, Ig-like lectins (Siglecs). The term “MAG” as used herein encompasses active glycosylated and non-glycosylated forms of MAG, active and non-active truncated forms or fragments of the molecule, and active larger peptides comprising SEQ ID NO:1. The term MAG is intended to include peptides comprising SEQ ID NO:1 that have been post-translationally modified. Post-translationally modified MAG includes phosphorylation, glycosylation, acylation, and proteolysis. In some embodiments, glycosylated MAG encompassed by the present invention comprises glycosylation at at least two sites for N-linked glycosylation, or in certain embodiments contains glycosylation at least three sties for N-linked glycosylation, or in other embodiments contains glycosylation at least four sites for N-linked glycosylation, or in other embodiments contains glycosylation at least five sites for N-linked glycosylation, or in other embodiments contains glycosylation at least six sites for N-linked glycosylation, or in other embodiments contains glycosylation at least seven sites for N-linked glycosylation, or in other embodiments contains glycosylation at least eight sites for N-linked glycosylation. In some embodiments, glycosylated MAG encompassed by the present invention comprises at least about 3% by weight carbohydrate, or in other embodiments contains at least about 6% by weight carbohydrate, or in other embodiments contains at least about 9% by weight carbohydrate, or in other embodiments contains at least about 15% by weight carbohydrate, or in other embodiments contains at least about 20% by weight carbohydrate, or in other embodiments contains at least about 25% by weight carbohydrate, or in other embodiments contains at least about 29% by weight carbohydrate.

In the present invention, the terms “fragments” or “truncations” are used interchangeably to mean a chemical substance that is related structurally and functionally to another substance. A fragment or truncation contains a modified structure from the parent substance, in this instance, at least one Ig domain of MAG and/or the biological function or activity of MAG in cellular and animal models. Possible functions assigned to MAG based on its subcellular location, biochemical properties and phenotypical properties of MAG-deficient mice include initiation and progression of myelination, cell adhesion events, such as through binding to sialic acid epitopes on other cells and integrin binging, membrane motility, endocytosis, signal transduction inside the glial cell an between the glial cell and the neuron, and inhibition of neurite outgrowth and axonal regeneration. Non-limiting examples of in vitro biological assays for MAG, including the neurite outgrowth inhibition assay and binding assay, are described in Example 4. Fragments of MAG can be less than about 626 amino acids in length and are substantially homologous to SEQ ID NO: 1. In other embodiments, fragments of MAG can be less than about 517 amino acids in length, less than about 326 amino acids in length, less than about 241 amino acids in length, or less than about 139 amino acids in length. In some embodiments, fragments of MAG include an affinity tag, including, but not limited to a polyhistidine tail or a FLAG tag (e.g., amino acid sequence DYKDDDDK) at the C-terminus or N-terminus.

As used herein, two polypeptides are “substantially homologous” when there is at least 70% homology, at least 80% homology, at least 90% homology, at least 95% homology or at least 99% homology between their amino acid sequences, or when polynucleotides encoding the polypeptides are capable of forming a stable duplex with each other. Likewise, two polynucleotides are “substantially homologous” when there is at least 70% homology, at least 80% homology, at least 90% homology, at least 95% homology or at least 99% homology between their amino acid sequences or when the polynucleotides are capable of forming a stable duplex with each other. In general, “homology” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis of similarity and identity, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence similarity and identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent similarity of a particular nucleotide sequence to a reference sequence can be determined using the homology algorithm of Smith and Waterman with a default scoring table and a gap penalty of six nucleotide positions. Alternatively, homology can be determined by hybridization of polynucleotides under conditions that form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As used herein, the term “metal affinity resin” includes, but is not limited to: resins containing an immobilized functional moiety (e.g. iminodiacetic acid) capable of binding and coordinating multivalent cations including Chelating-Sepharose, Fractogel-EMD-Chelate, POROS 20 MC, and Matrex Cellufine Chelate. The bound metal ion can be selected from several possible choices including but not limited to copper, nickel, cadmium, calcium, cobalt, iron, zinc, or strontium.

I. MAG and Truncations Thereof

Nerve regeneration is an important step for the development of novel therapies for human conditions derived from axonal damage in the central nervous system (CNS). Myelin-associated glycoprotein (MAG) is a transmembrane cell adhesion molecu that is an inhibitor of axon regeneration and has an important role in maintaining a stable interaction between axons and myelin. MAG also plays a role in a number of neurodegenerative diseases. For example, early loss of MAG in the development of multiple sclerosis plaques suggests a role in the pathogenesis of this disease. MAG functions in glia-axon interactions in both the peripheral nervous system (PNS) and the central nervous system (CNS) and is expressed by myelinating glial cells (Quarles R H, et al. (1972) Biochem Biophys Res Commun 47: 491-497). It is a member of the sialic acid binding subgroup of the immunoglobulin superfamily and shares significant homology with the neural cell adhesion molecule (N-CAM).

In one aspect of the invention, DNA sequences are provided which include: the incorporation of codons preferred for expression by selected nonmammalian hosts; the provision of sites for cleavage by restriction endonuclease enzymes; and the provision of additional initial, terminal or intermediate DNA sequences which facilitate construction of readily-expressed vectors. The present invention also provides DNA sequences coding for polypeptide analogs or derivatives of MAG which differ from naturally-occurring forms in terms of the identity or location of one or more amino acid residues (i.e., deletion analogs containing less than all of the residues specified for MAG; substitution analogs, wherein one or more residues specified are replaced by other residues; and addition analogs wherein one or more amino acid residues is added to a terminal or medial portion of the polypeptide) and which share some or all the properties of naturally-occurring forms.

Novel DNA sequences of the invention include sequences useful in securing expression in prokaryotic or eukaryotic host cells of polypeptide products having at least a part of the primary structural conformation and one or more of the biological properties of naturally-occurring MAG. Non-limiting DNA sequences of the invention specifically comprise: (a) DNA sequences set forth in SEQ ID Nos. 4-6 or their complementary strands; (b) DNA sequences which hybridize to the DNA sequences in SEQ ID Nos. 4-6 or to fragments thereof; and (c) DNA sequences which, but for the degeneracy of the genetic code, would hybridize to the DNA sequences in SEQ ID Nos. 4-6. Specifically comprehended in parts (b) and (c) are genomic DNA sequences encoding allelic variant forms of MAG and/or encoding MAG from other mammalian species, and manufactured DNA sequences encoding MAG, fragments of MAG, and analogs of MAG. The DNA sequences may incorporate codons facilitating transcription and translation of messenger RNA in microbial hosts. Such manufactured sequences may readily be constructed according to the methods of Alton et al., PCT published application WO 83/04053.

According to another aspect of the present invention, the DNA sequences described herein which encode polypeptides having MAG activity are valuable for the information which they provide concerning the amino acid sequence of the mammalian protein which have heretofore been unavailable. The DNA sequences are also valuable as products useful in effecting the large scale synthesis of MAG by a variety of recombinant techniques. DNA sequences provided by the invention are useful in generating new and useful viral and circular plasmid DNA vectors, new and useful transformed and transfected prokaryotic and eukaryotic host cells (including bacterial and yeast cells and mammalian cells grown in culture), and new and useful methods for cultured growth of such host cells capable of expression of MAG and its related products.

The present invention provides purified and isolated naturally-occurring MAG such that the primary, secondary and tertiary conformation, and the glycosylation pattern are substantially identical to naturally-occurring material, as well as non-naturally occurring MAG fragments having a primary structural conformation (i.e., continuous sequence of amino acid residues) and glycosylation substantially duplicative of that of naturally occurring MAG to allow possession of a neurite outgrowth inhibitory activity substantially similar to that of naturally occurring MAG (See Example 4).

In a certain embodiment, recombinant MAG is produced as a product of prokaryotic or eukaryotic host expression (e.g., by bacterial, yeast, higher plant, insect and mammalian cells in culture) of exogenous DNA sequences obtained by genomic or cDNA cloning or by gene synthesis. The products of expression in typical yeast (e.g., Saccharomyces cerevisiae) or prokaryote (e.g., E. coli) host cells are free of association with any mammalian proteins. The products of expression in vertebrate [e.g., non-human mammalian (e.g. COS or CHO) and avian] cells are free of association with any human proteins. Depending upon the host employed, polypeptides of the invention may be glycosylated with mammalian or other eukaryotic carbohydrates or may be non-glycosylated. The host cell can be altered using techniques such as those described in Lee et al. J. Biol. Chem. 264, 13848 (1989) hereby incorporated by reference. Polypeptides of the invention may also include an initial methionine amino acid residue (at position −1).

In addition to naturally-occurring allelic forms of MAG, the present invention also embraces other MAG fragments such as polypeptide analogs of MAG. Such analogs include fragments of MAG. One of ordinary skill in the art can readily design and manufacture genes coding for expression of polypeptides having primary conformations which differ from that herein specified for in terms of the identity or location of one or more residues (e.g., substitutions, terminal and intermediate additions and deletions) (See, for example procedures, Alton et al. (WO 83/04053)). Alternately, modifications of cDNA and genomic genes can be readily accomplished by well-known site-directed mutagenesis techniques and employed to generate analogs and derivatives of MAG. Such products share at least one of the biological properties of MAG but may differ in others. As non-limiting examples, products of the invention include those which are foreshortened by e.g., deletions; or those which are more stable to hydrolysis (and, therefore, may have more pronounced or longer-lasting effects than naturally-occurring); or which have been altered to delete or to add one or more potential sites for O-glycosylation and/or N-glycosylation or which have one or more cysteine residues deleted or replaced by, e.g., alanine or serine residues and are potentially more easily isolated in active form from microbial systems; or which have one or more tyrosine residues replaced by phenylalanine and bind more or less readily to target proteins or to receptors on target cells. Also comprehended are polypeptide fragments duplicating only a part of the continuous amino acid sequence or secondary conformations within MAG, which fragments may possess one property of MAG (e.g., receptor binding) and not others (e.g., neurite outgrowth inhibition). It is noteworthy that activity is not necessary for any one or more of the products of the invention to have therapeutic utility or utility in other contexts, such as in assays of MAG antagonism. Competitive antagonists may be useful to, for example, block the inhibitory affect of MAG.

The present invention also includes that class of polypeptides coded for by portions of the DNA complementary to the protein-coding strand of the human cDNA or genomic DNA sequences of MAG, i.e., “complementary inverted proteins.”

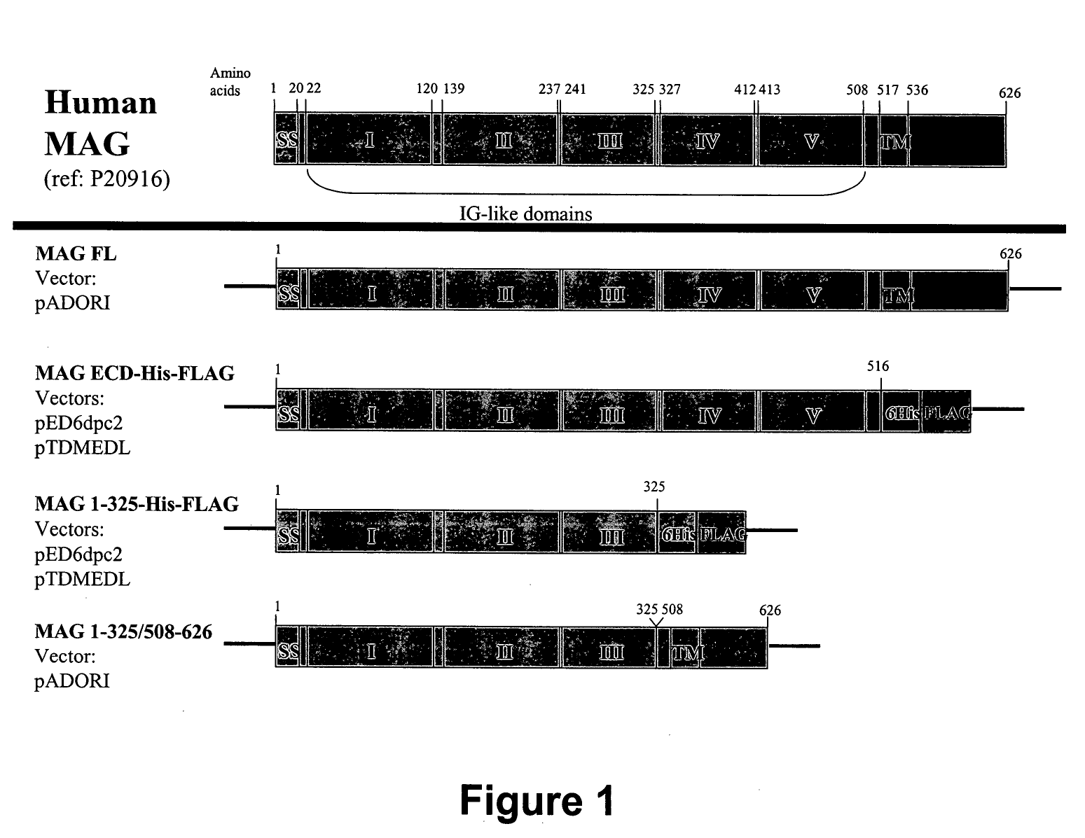

In one aspect of the invention, MAG fragments can be designed comprising one, two, three, four or five Ig domains based on the amino acid sequence assigned to each Ig domain shown in FIG. 1. Primers are designed as well-known in the art to allow for proper expression of each domain. To allow for proper secondary structure, primers can be designed such that the fragment extends into the adjacent Ig domain. For example, a MAG fragment comprising the first three IG domains of MAG can be designed to comprise amino acid residues at least amino acids 1-325, at least amino acids 1-327, or at least amino acids 1-350, at least amino acids 1-375. In another aspect of the invention, MAG fragments can be constructed to fuse non-adjacent MAG Ig domains. For example, amino acids 1-325 comprising Ig domains 1-3 can be fused to amino acids 413-508 comprising Ig domain 5.

Representative MAG polypeptides of the present invention include, but are not limited to, MAG1-120, MAG1-237, MAG1-325, MAG1-412, MAG1-508, MAG1-517, MAG1-536, MAG1-626, MAG1-139, MAG1-241, MAG1-327, MAG1-413, MAG1-160, MAG1-180, MAG1-200, MAG1-260, MAG1-280, MAG1-300, MAG1-350, MAG1-370, MAG1-390, MAG1-430, MAG1-450, MAG1-470, MAG20-120, MAG237-327, MAG325-413, MAG412-508, MAG412-516, MAG325-516, MAG90-260, MAG210-340, MAG310-430, MAG390-516, MAG390-626, MAG1-241, MAG120-327, MAG120-413, MAG120-516, MAG120-626, MAG237-413, MAG237-516, MAG237-536, MAG237-626, MAG325-508, MAG325-536, MAG325-626, MAG1-325/508-626. The MAG fragments of the present invention can include a polyhistidine tag (i.e., His6) and/or a FLAG tag at either the C-terminus or N-terminus.

II. MAG Purification

In one aspect, the present invention comprises a one-step method of purifying MAG and MAG fragments from a MAG containing material such as conditioned medium.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods, which are well known to those skilled in the art, may be used to construct expression vectors containing sequences encoding a polypeptide of interest and appropriate transcriptional and translational control elements.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. However, the use of a bacterial system does not allow for post-translational modification substantially identical to the endogenous MAG protein. Furthermore, the use of a fusion protein requires a multi-step purification process.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus that is capable of expressing the polypeptide in infected host cells. In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However; in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature.

In general, a DNA sequence encoding a MAG polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers can be provided on separate vectors, and replication of the exogenous DNA is provided by integration into the host cell genome. Selection of promoters, terminators, selectable markers, vectors and other elements is a matter of routine design within the level of ordinary skill in the art. Many such elements are described in the literature and are available through commercial suppliers.

To direct a MAG polypeptide into the secretory pathway of a host cell, a secretory signal sequence (also known as a leader sequence, prepro sequence or pre sequence) is provided in the expression vector. The secretory signal sequence may be derived from another secreted protein or synthesized de novo. The secretory signal sequence is operably linked to the MAG DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest.

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and W138, which have specific cellular machinery and characteristic mechanisms for such post-translational activities, may be chosen to ensure the correct modification and processing of the foreign protein.

Cultured mammalian cells are preferably used as host cells in the present invention. Methods for introducing exogenous DNA into mammalian host cells include calcium phosphate-mediated transfection, DEAE-dextran mediated transfection, and liposome-mediated transfection. The production of recombinant polypeptides in cultured mammalian cells is well known in the art. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1, ATCC No. CCL 61; or CHO DG44, Chasin et al., Som. Cell. Molec. Genet. 12:555, 1986) cell lines. Additional suitable cell lines are known in the art and available from public depositories such as the American Type Culture Collection, Manassas, Va. Suitable promoters include those from metallothionein genes, the adenovirus major late promoter, and promoters from SV-40 or cytomegalovirus.

For long-term, high-yield production of recombinant proteins, stable expression is generally preferred. For example, cell lines that stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. Antimetabolite, antibiotic or herbicide resistance can be used as the basis for selection; for example, dhfr which confers resistance to methotrexate; npt, which confers resistance to the aminoglycosides, neomycin and G-418; and als or pat, which confer resistance to chlorsulfuron and phosphinotricin acetyltransferase, respectively. Additional selectable genes have been described, for example, trpB, which allows cells to utilize indole in place of tryptophan, or hisD, which allows cells to utilize histinol in place of histidine. The use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system.

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences that direct secretion of the encoded polypeptide through a prokaryotic or eukaryotic cell membrane. Other recombinant constructions may be used to join sequences encoding a polypeptide of interest to nucleotide sequence encoding a polypeptide domain that will facilitate purification of soluble proteins. Such purification facilitating domains include, but are not limited to, metal chelating peptides such as histidine-tryptophan modules that allow purification on metals affinity resins, and the domain utilized in the FLAGS extension/affinity purification system (Immunex Corp., Seattle, Wash.).

In addition to recombinant production methods, MAG of the invention, and fragments thereof, may be produced by direct peptide synthesis using solid-phase techniques. Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Recombinant MAG and MAG fragments can be extracted from the spent culture medium using combinations of centrifugation, ultrafiltration and chromatography. In a certain embodiment, culture medium from the CHO cells is passed over a charged metal affinity resin. The metal affinity resin can be charged by passing a solution of the metal salt over the column packed with uncharged chelating matrix. The pH will affect the protein binding. Additional reagents such as urea, salts, or detergents may be added to the binding buffer. The bound MAG fragments should be washed thoroughly and then can be eluted from the metal affinity resin using several methods, such as through the use of a pH gradient, the use of a competitive ligand, such as imidazole, histamine, glycine, or ammonium chloride, or the use of a chelating agent such as EDTA or EGTA.

Measurement of the relative amount of purified MAG or MAG fragment may be by any method known in the art. Typical methodologies for protein detection include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis,-column chromatography, direct sequencing, or quantitative PCR (in the case of polynucleotides) among many other techniques well known to those skilled in the art.

The MAG proteins of the invention can be measured by mass spectrometry, which allows direct measurements with high sensitivity and reproducibility. A number of mass spectrometric methods are available. Electrospray ionization (ESI), for example, allows quantification of differences in relative concentration of various species in one sample against another; absolute quantification is possible by normalization techniques (e.g., using an internal standard). Mass spectrometers that allow time-of-flight (TOF) measurements have high accuracy and resolution and are able to measure low abundant species.

The compositions and methods of the invention are demonstrated in the Examples. The purification process of the present invention is demonstrated using two versions of myelin associated glycoprotein (MAG), MAG(1-3) (SEQ ID NO: 3) and MAG(1-5) (SEQ ID NO: 2). Using a battery of techniques, the purity and bioactivity of the purified MAG(1-3) and MAG(1-5) were confirmed. The effect of storage conditions and handling methods of the present invention on the stability of the product under various conditions is demonstrated.

III. Uses of Purified MAG and Fragments Thereof

In one aspect the methods and constructs of the present invention may have diagnostic and/or therapeutic use in neurological disorders. The terms “neurological disorder” or “CNS disorder,” refer to an impairment or absence of a normal neurological function or presence of an abnormal neurological function in a subject. For example, neurological disorders can be the result of disease, injury, and/or aging. As used herein, neurological disorder also includes neurodegeneration, which causes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurological disorders include, but are not limited to, memory disorders, dementia, memory loss, epilepsy, and ischemia. Neurological disorders also include neurodegenerative diseases. Neurodegeneration can occur in any area of the brain of a subject and is seen with many disorders including, but not limited to, Alzheimer's Disease, Amyotrophic Lateral Sclerosis (ALS), multiple sclerosis, Huntington's disease, and Parkinson's disease.

Molecules capable of specifically binding to MAG and that block MAG's inhibitory function are included within the invention. In some embodiments, the binding molecules are antibodies or antibody fragments. In other embodiments, the binding molecules are non-antibody species. Thus, for example, the binding molecule may be an enzyme for which MAG is a substrate. The binding molecules may recognize any epitope of MAG. The binding molecules may be identified and produced by any method accepted in the art. Methods for identifying and producing antibodies and antibody fragments specific for an analyte are well known. Examples of other methods used to identify the binding molecules include binding assays with random peptide libraries (e.g., phage display) and design methods based on an analysis of the structure of the MAG.

In another aspect, MAG and MAG fragments of the present invention can be used to explore structure-function analysis of MAG receptors, including, but not limited to, NOGO receptor (NgR) and p75(NTR), and LINGO-1. Using the novel fragments of the invention, specific domain-domain interactions can be explored and can lead to the development of novel therapeutics.

In another aspect of the invention, the inhibitory function of MAG proteins and/or MAG receptors could be blocked with antibodies or peptides. Recombinant MAG protein and fragments thereof of the present invention can be used as immunogens or selection targets in generating MAG-specific antibodies. In addition, the recombinant MAG protein and fragments thereof of the present invention can be used in assays for studying NoGo receptor interactions with its ligands as well as in development of therapeutic agents blocking interactions for treatment of spinal cord injuries, cerebral ischemic injuries, and neurological disorder.

Axonal regeneration in the adult CNS is limited by at least three proteins in myelin, myelin-associated glycoprotein (MAG), Nogo, and oligodendrocyte myelin glycoprotein (Omgp). The NOGO receptor (NgR) had been identified as an axonal GPI-anchored protein, whereas the MAG receptor had remained elusive. MAG has been shown to bind directly, with high affinity, to NgR (Liu, B. P. et al. Myelin-associated glycoprotein as a functional ligand for the Nogo-66 receptor. Science 297: 1190-1193, 2002.). Cleavage of GPI-linked proteins from axons protects growth cones from MAG-induced collapse, and dominant-negative NgR eliminates MAG inhibition of neurite outgrowth. MAG-resistant embryonic neurons were rendered MAG-sensitive by expression of NgR. MAG and Nogo-66 activate NgR independently and serve as redundant NgR ligands that may limit axonal regeneration after CNS injury.

Recently, it was shown that MAG inhibits axonal regeneration through interaction with NgR (Domeniconi, M. et al. Myelin-associated glycoprotein interacts with the Nogo66 receptor to inhibit neurite outgrowth. Neuron 35: 283-290, 2002). MAG binds specifically to an NgR-expressing cell line in a GPI-dependent and sialic acid-independent manner. MAG precipitates NgR from NgR-expressing cells, dorsal root ganglia, and cerebellar neurons which is consistent with a direct interaction of MAG and NgR. Experiments blocking NgR from interacting with MAG prevented inhibition of neurite outgrowth by MAG. MAG and NOGO-66 compete directly for binding to NgR (Domeniconi, M. et al. Neuron 35: 283-290, 2002).

In inhibiting neurite outgrowth, several myelin components, including the extracellular domain of NOGOA, OMGP, and MAG, exert their effects through the same NOGO receptor. The glycosylphosphatidylinositol (GPI)-anchored nature of the NOGO receptor indicates the requirement for an additional transmembrane protein to transduce the inhibitory signals into the interior of responding neurons. p75, a transmembrane protein known to be a receptor for the neurotrophin family of growth factors, specifically interacts with the NOGO receptor. p75 is required for NOGO receptor-mediated signaling, as neurons from p75 knockout mice were no longer responsive to myelin or to any of the known NOGO receptor ligands. Blocking the p75-NOGO receptor interaction also reduced the activities of these inhibitors. Moreover, a truncated p75 protein lacking the intracellular domain, when overexpressed in primary neurons, attenuated the same set of inhibitory activities, suggesting that p75 is a signal transducer of the Nogo receptor-p75 receptor complex. Interfering with p75 and its downstream signaling pathways may allow lesioned axons to overcome most of the inhibitory activities associated with central nervous system myelin.

In another aspect, the inhibitory function of MAG can be blocked with antibodies or peptides that bind to p75. p75(NTR) is a coreceptor for the NOGO receptor for MAG signaling (Wong, S. T.; et al. A p75(NTR) and Nogo receptor complex mediates repulsive signaling by myelin-associated glycoprotein. Nature Neurosci. 5: 1302-1308, 2002). In cultured human embryonic kidney (HEK) cells expressing the NOGO receptor, p75(NTR) was required for MAG-induced intracellular calcium elevation. Coimmunoprecipitation showed an association of the NOGO receptor with p75(NTR) that could be disrupted by an antibody against p75(NTR), and extensive coexpression was observed in the developing rat nervous system. Furthermore, a p75(NTR) antibody abolished MAG-induced repulsive turning of Xenopus axonal growth cones and calcium elevation, both in neurons and in the NOGO receptor/p75(NTR)-expressing HEK cells. In another aspect of the invention, the MAG vector can be used as a genetic vaccine. DNA encoding the full-length human MAG ORF, in an adenoviral vector, can be used directly in the gene gun to immunize a subject. The MAG protein is expressed on the surface of cells that receive DNA from the gene gun. In in vitro transient expression assays, protein could be detected on cell surface. In addition, human MAG Ig-like domains I-III fused to TM-ICD in an adenoviral vector can be used directly in the gene gun to immunize a subject. MAG domain I-III protein will be expressed on cell surface. All MAG fragments disclosed in this invention can be expressed in an adenoviral vector and used as a genetic vaccine.

### EXAMPLES

The following examples illustrate practice of the invention. These examples are for illustrative purposes only and are not intended in any way to limit the scope of the invention claimed.

Myelin associated glycoprotein (MAG) is a membrane-bound cell adhesion molecule and belongs to the IgG-gene super family. It consists of five extracellular IgG-like domains with multiple functions in myelin formation and maintenance. MAG interacts with its receptors on neurons producing neurite collapse and inhibition of axonal regeneration. To demonstrate the methods of the present invention, the expression and purification of different versions of recombinant human MAG were explored. Examples 1-3 detail the construction of vectors, expression and purification of MAG(1-3) (amino acids 1-325, SEQ ID 3 and SEQ ID 6) and MAG(1-5) (amino acids 1-516, SEQ ID NO: 2 and SEQ ID NO: 5) fused to His6 and FLAG tags at the C-termini which were expressed in stably transfected CHO cell lines. Cells were cultured to confluence in a defined medium containing 10% FBS and 100 nM methotrexate, and switched to the defined medium without serum supplement 48 hours prior to harvesting. Purification techniques using various metal affinity columns are disclosed. The purified products were characterized using various techniques. Efficient storage and handling conditions that retain stability and function of the purified protein are described in Example 5.

### Example 1

### Generation of MAG Proteins and Fragments.

To generate MAG and MAG fragments, IMAGE consortium clone (5194207) comprising a full-length open reading encoding amino acids corresponding to those of GenBank Accession No. P20916 (SEQ ID NO: 1) was used as a template for PCR amplification with the following primers:

1) pADORI-MAG FL Clone aa 1-626

(SEQ ID NO. 7)

| | | |
|---|---|---|
| 5′ oligo: | 5′gatcgatcagatctgccgccatgatat | BglII site |
| | tcctcacggcact | |

(SEQ ID NO. 8)

| | | |
|---|---|---|
| 3′oligo: | 5′tagtactagaattctcatcacttgacc | EcoRI site |
| | cggatttcagcatactca | |

2) pED6 and pTDMEDL-MAG-ECD 6His-FLAG (MAG aa 1-516):

(SEQ ID NO. 9)

| | | |
|---|---|---|
| 5′ oligo: | 5′gatcgatctctagagccgccatgatat | XbaI site |
| | tcctcacggcact | |

(SEQ ID NO. 10)

| | | |
|---|---|---|
| 3′oligo: | 5′tagtactagaattctcatcagatctta | EcoRI site |
| | tcgtcgtcatccttgtaatcatggtgatg | |
| | atggtgatgaggcccgatcttggccc | |
| | acat | |

3) pED6 and pTDMEDL-MAG-I-III 6His-FLAG (MAG aa 1-325):

(SEQ ID NO. 11)

| | | |
|---|---|---|
| 5′ oligo: | 5′gatcgatctctagagccgccatgatatt | XbaI site |
| | cctcacggcact | |

(SEQ ID NO. 12)

| | | |
|---|---|---|
| 3′oligo: | 5′tagtactagaattctcatcagatcttat | EcoRI site |
| | cgtcgtcatccttgtaatcatggtgatgat | |
| | ggtgatgtgcatacatgacactgagccc | |

4) pADORI-MAG aa 1-325/508-626:

Primer Set 1:

(SEQ ID NO. 13)

| | |
|---|---|
| oligo A (5′-3′): | tcagcgatcactcactcgctgtacaga |

(SEQ ID NO. 14)

| | |
|---|---|
| oligo B: | aggcccgatcttggcccacatcagtcgtgcata |
| | catgacactgagccccac |

Primer Set 2:

(SEQ ID NO. 15)

| | |
|---|---|
| OligoC: | ggggctcagtgtcatgtatgcacgactgatgtgggccaagatc |
| | ggg |

(SEQ ID NO. 16)

| | |
|---|---|
| Oligo D: | gggtcagccacgcaggctgcccccagctcct |

Primer Set 3:

(SEQ ID NO. 17)

| | |
|---|---|
| Oligo A′: | 5′gatcgatcagatctgccgccatgatattcctcacggc |
| | act |

(SEQ ID NO. 18)

| | |
|---|---|
| Oligo D′: | gatcgatcgaattctcatcacttgacccggatttcagcat |
| | actc |

FIG. 1 identifies the amino acids comprising each of the five Ig domains. One skilled in the art will be able to design primers such that the resulting MAG fragments comprise one, two, three, four or five Ig domains using the DNA sequence disclosed in SEQ ID NOS: 4-6. In some instances, it may be beneficial to design primers such that the sequence extends into the adjacent Ig domain to allow for proper secondary structure.

The generation and purification of several MAG fragments are detailed below as representative examples of the above invention. DNA was amplified from an IMAGE consortium clone (5194207). A partial sequence of this clone can be found in Genbank Accession No. BI755065. The clone contained a full-length open reading with amino acid numbers corresponding to those of GenBank Accession No. P20916 (SEQ ID NO: 1). To construct MAG(1-5) vectors, DNA encoding residues 1-516 was amplified, fused to 6His-FLAG tag sequence and ligated in frame with pED6 and pTMEDL (CHO cell and COS cell expression vectors) using XbaI/EcoRI sites. To construct MAG(1-3) expression vectors, DNA encoding amino acid (aa) residues 1-325 was amplified, fused to 6His-FLAG tag sequence and ligated in frame with pED6 and pTMEDL (CHO cell and COS cell expression vectors) using XbaI/EcoRI sites. To construct a MAG-FL expression vector, DNA encoding aa residues 1-626 was amplified, and ligated in frame with pADORI (adenoviral vector) using BglII/EcoRI sites. To construct a MAG-(1-3/TM-ICD) expression vector, DNA encoding aa residues 1-325 were fused to amino acid residues 508-626 comprising the transmembrane (TM) and intracellular domain (ICD) of human MAG and cloned into BglII/EcoRI sites of pADORI (adenoviral vector). MAG-(1-3/TM-ICD) was constructed with two PCR amplifications using IMAGE consortium clone (5194207) as a template. Primer set 1 and primer set 2 (shown above) were used to generate PCR reaction products 1 and 2. Next, PCR was used to amplify the pooled reaction products 1 and 2 using primer set 3, generating reaction product 3. Reaction product 3 was digested with BglII/EcoRI and cloned into pAdori1-3 cut with BglII/EcoRI. All MAG proteins were produced by stable transfection of CHO cells and purified using various metal affinity columns as described below.

### Example 2

### Expression of Recombinant MAG in Chinese Hamster Ovary (CHO) Cells

This example relates to a stable mammalian expression system for secretion of MAG from CHO cells.

For stable expression in CHO cells, the CHO cell vectors comprising the human MAG fragments MAG(1-3) (amino acids 1-325, SEQ ID NO: 2) and MAG(1-5) (amino acids 1-516, SEQ ID NO: 3) fused to His6 and FLAG tags at the C-termini detailed above in Example 1 were transfected into duplicate 100 mm plates using TransIT-CHO Transfection Kit, (Cat #: MIR 2170 from Mirus Corporation Madison, Wis. 53719-1267 USA) and using the protocol that the kit provided. CHO cells were transfected with the MAG-TMED plasmid containing a selectable marker, the DHFR gene. Methotrexate was added to the media to select for transfected CHO cells. As a control, the vector without insert was also transfected.

After 24 hour transfection, MAG transfected cells and vector transfected cells were split from the duplicate 100 mm plates to 6 of 100 mm plates. Cultured with a Alpha medium (Wyeth)+10% dialyzed heat-inactivated FBS (Gibco, USA Cat #: 26400-044) and Penicillin-Streptomycin (PenStrep Gibco US cat #:15070-063)/ L-Glutamine (Gibco USA, cat # 25030-140). Each two plates added different concentrations of selection drug methotrexate (MTX, Sigma, Ga., USA; Cat #: M 9929): 20 nM (MTX), or 50 nM MTX, or 100 nM MTX. The plates were incubated at 37° C., 5% CO₂. Once the colonies formed and looked large and healthy enough (approx. two weeks), single colonies were picked and placed into a 96-well plate containing 150 μl of 50 nM MTX selection medium, from which 25 μl was transferred to a 24 well plate as a live cell bank. No colonies were formed from vector transfected cells in different concentration selection medium or from MAG transfected cells in 100 nM MTX medium.

Cells in 96-well plate were cultured to about confluence, then switched to the R5 CD1 medium (Wyeth) without serum supplement 48 hours prior to harvesting. Conditioned media (CM) was run on a 4-20% SDS gel and analyzed by Western blot with anti-MAG antibody (Cat #: sc-1 5324 anti-MAG (H-300) rabbit polyclonal Santa Cruz Biotechnology USA) to select the higher expression clones. The selected clones from the 24 well plate cell bank were split into 100 nM MTX selection medium, cultured to about confluence, then switched to the R5 CD1 medium without serum supplement 48 hours prior to harvesting. CM run on a 4-20% SDS gel and analyzed by Western blot with Anti-MAG antibody again. The clones that secreted higher amounts of protein, were healthy and grew faster were chosen as stable cell lines. Selected stable cell lines were maintained at 100 nM MTX and 10% dialyzed FBS alpha medium with PenStrep/Glutamine.

### Example 3

### Purification of MAG Proteins and Fragments

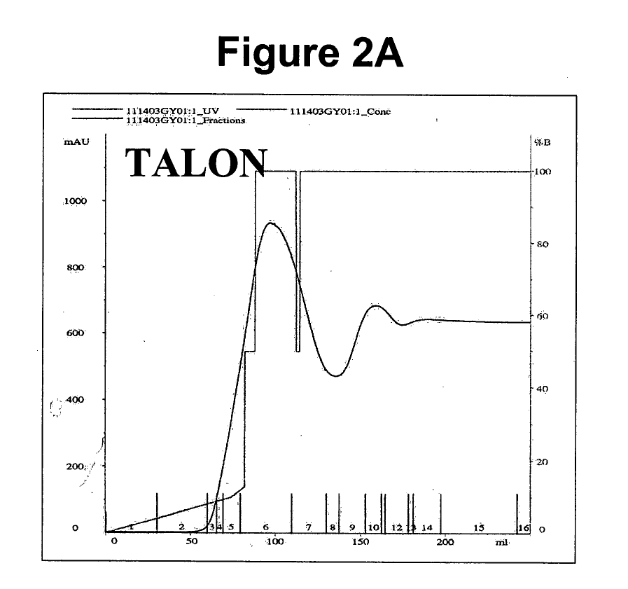

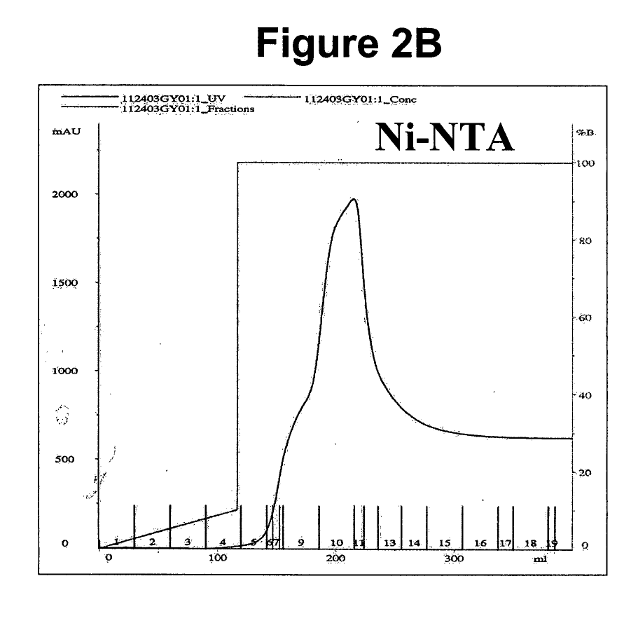

Upon harvesting the conditioned media from the CHO cells, the media can be filtered through a 0.2 uM filter and NaAzide can be added to 0.01%. The pH of the conditioned media was adjusted to around 8.0 using 2M Tris, pH 8.5 and loaded onto the HPLC with either a Nickel column (Ni—NTA, Qiagen, Calif.) or cobalt column (TALON™, BD Biosciences Clontech, Canada) with a flowrate of 2-4 ml/min. The column was washed and the bound protein was eluted at a flowrate of 8 ml/min using the following gradient: 0-10% Buffer B in 1.5 column volumes (cv), 50% Buffer B for 0.1 cv, 100% Buffer B for 5 cv where Buffer A is 300 mM NaCl, 50 mM Na₂HPO₄, pH 8.0 and Buffer B is 500 mM Imidazole A, 300 mM NaCl, 50 mM Na2HPO4, pH 8.0. Purification chromatograms representative of the TALON™ and Ni—NTA column purification of MAG1-5 are shown in FIGS. 2A and 2B, respectively.

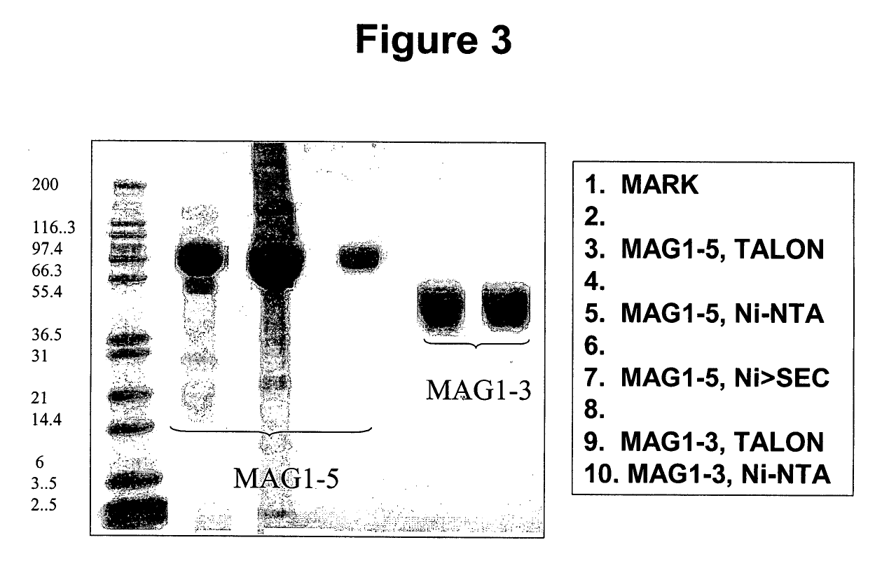

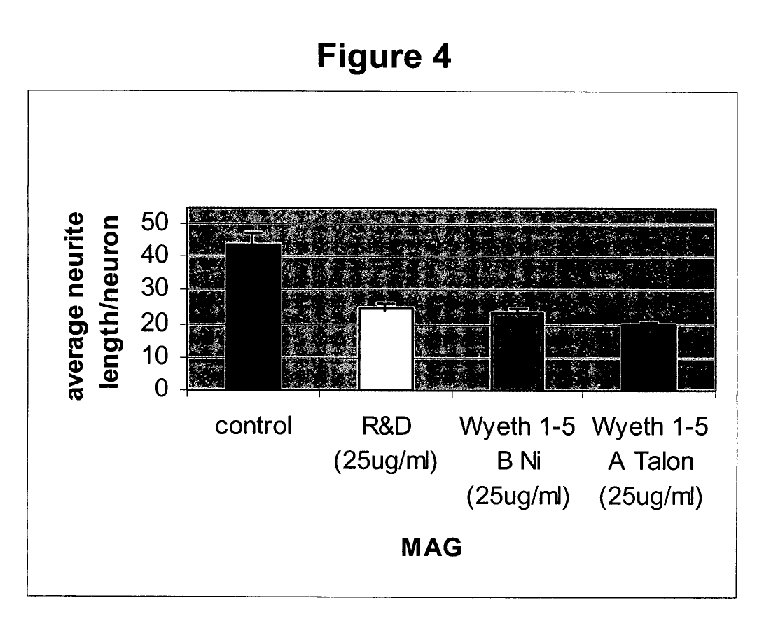

SDS PAGE was used to evaluate the purity of the eluted protein. FIG. 3 shows one dominant band of MAG1-3 purified from both TALON™ and Ni—NTA column purification. SDS PAGE confirms that the purified MAG1-5 has only a single major band.

The purity of MAG(1-5) and MAG(1-3) were confirmed through the additional characterization which included N-terminal sequencing, Western blot analysis, LC/MS, size exclusion chromatography (SEC), isoelectric focusing (IEF), and UV analysis. SEC confirmed that purified MAG(1-5) has a purity >96%. LC/MS confirmed CHO proteins are the major contaminant proteins of purified MAG(1-5) and purified MAG(1-3). IEF found that the PI of MAG(1-5) is 4.4, which is substantially the same as the reported value. Western blot confirmed both major and the minor band underneath are MAG(1-3). SEC confirmed MAG(1-3) has a purity >99%.

### Example 4

### Biological Assays of MAG Proteins and Fragments

i) In vitro binding assay. MAG immobilized on Ni²⁺⁺ resin was incubated with buffer alone or with NgR-ecto (residues 27-310) in the presence of 1% BSA for 1 hour and bound protein was eluted with 500 mM imidazole. NgR-ecto was also incubated with Ni²⁺⁺ resin alone to rule out nonspecific binding to the affinity resin. (See protocol described in Liu et al. Science, 297: 1190-1193 (2002).

ii) Neurite outgrowth assays. To test the inhibitory effect of purified MAG and MAG fragments, dissociated neurons can be plated on increasing concentration of inhibitory substrates (purified MAG fragments or Fc as a control). Neurons can then be grown for 4-8 hours, fixed, stained with rhodamine phalloidin, and neurite outgrowth lengths can be assessed using NIH image.

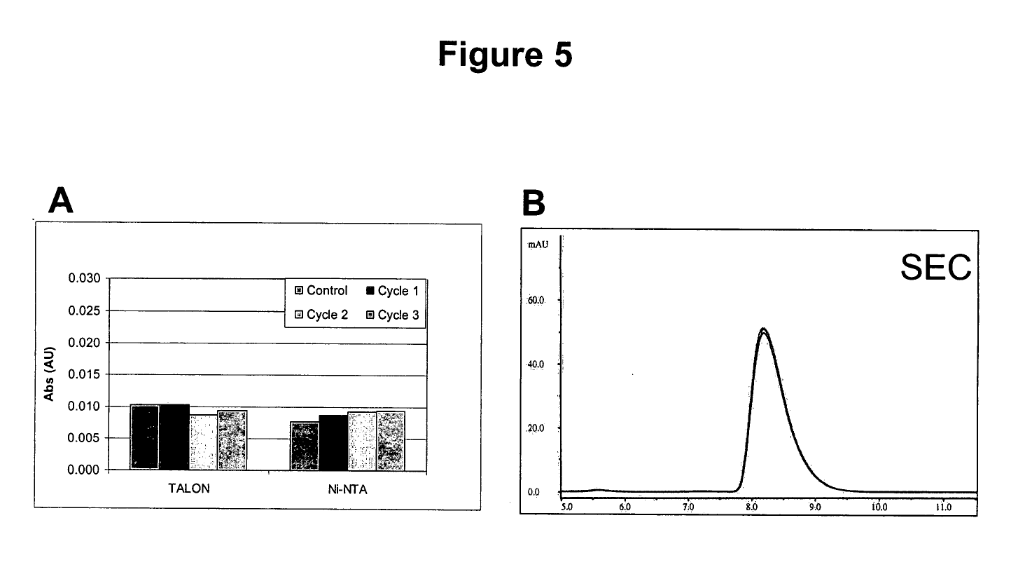

In other experiments, rat cerebellar granular neurons were treated with 25 ug/ml purified recombinant MAG or control (Fc domain) for 24 hrs. Neurons were grown on a monolayer of 3T3 cells and neurite length scored by manual analysis. FIG. 5 shows that MAG1-5 has about a 45-50% inhibition of neurite outgrowth. The results demonstrate that the purified MAG1-5 using either nickel or cobalt resin is as effective as, if not better than, commercially available Fc-MAG (cat. no. 538-MG- 100, R&D Systems, Minneapolis, Minn.) at inhibiting neurite outgrowth.

### Example 5

### Stability of MAG and MAG Fragments

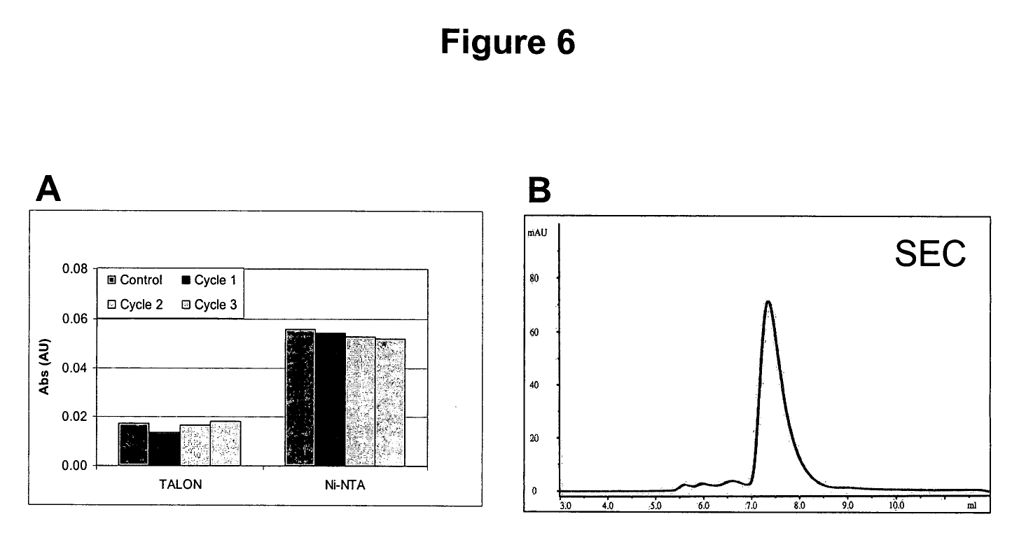

This Example demonstrates that both MAG1-3 and MAG1-5 are relatively stable. FIGS. 5A and 6A show that following three cycles of freeze/thaw from −80 ° C. to room temperature, there is no sign of protein destabilization, precipitation, or change in absorbance at 320 nm in either purified MAG1-3 or MAG1-5, respectively. SEC analysis further confirmed that there is no aggregation (See FIGS. 5B and 6B). Thus, multiple cycles of freezing and thawing have no effect on the stability of MAG(1-3) and MAG(1-5).

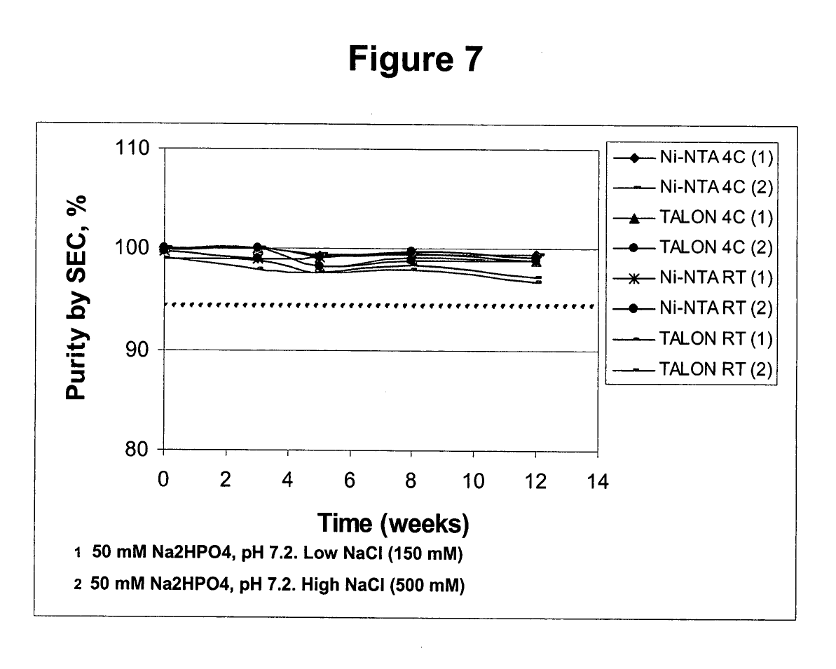

FIG. 7 demonstrates that the MAG1-3 is a very robust protein. The purity of MAG1-3 is not affected by storage temperature, the metal affinity resin or the salt concentration. FIG. 7 shows the % purity by SEC of MAG1-3 purified using either the Ni—NTA or TALON™ resin under various salt conditions [(1): 50 mM Na₂HPO₄, pH 7.2. Low NaCl (150 mM); (2): 50 mM Na₂HPO₄, pH 7.2. High NaCl (500 mM)] and stored at either room temperature or 4° C.

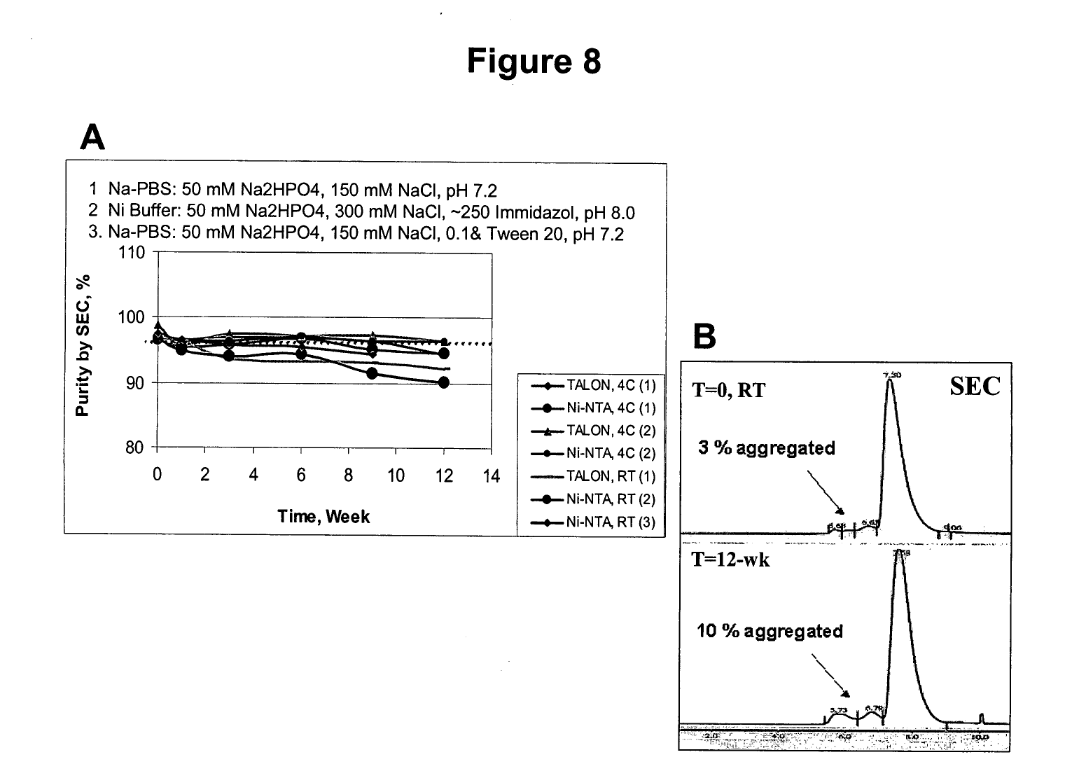

FIG. 8A demonstrates that MAG1-5 is affected slightly by buffer composition and storage temperature. Buffer comprising imidazole buffer is slightly better for stabilizing MAG1-5. Tween 20 has some limited effect in maintaining the stability of MAG1-5. FIG. 8B shows that aggregation increased when MAG1-5 was stored for 12 weeks at room temperature compared to storage at 4° C. FIG. 8 shows the % purity by SEC of MAG1-5 purified using either the Ni—NTA or TALON™ resin under various buffer conditions [(1): Na—PBS: 50 mM Na₂HPO₄, 150 mM NaCl, pH 7.2; (2): Ni Buffer: 50 mM Na₂HPO₄, 300 mM NaCl, ˜250 Immidazol, pH 8.0; (3): Na—PBS: 50 mM Na₂HPO₄, 150 mM NaCl, 0.1 & Tween 20, pH 7.2] and stored at either room temperature or 4° C.

In conclusion, metal affinity chromatography followed by size exclusion chromatography (SEC) is a good process for the purification of MAG1-3 and MAG1-5. Ni—NTA and TALON™ have no significant differences. Various characterization studies demonstrated that the both purified types of MAG have high purity and MAG1-5 has high bioreactivity. Multiple cycles of freezing and thawing has no effect on the stability of MAG1-3 and MAG1-5. MAG1-3 is stable when stored in Na—PBS at either 4 ° C. or ambient conditions over a 12 week period of time. The stability of MAG1-5 depends on the storage conditions. In the imidazole buffer, MAG1-5 is stable for at least 12 weeks when stored at 4° C. In Na—PBS, MAG1-5 is stable for 9 weeks when stored at 4° C., but when stored under ambient condition, MAG1-5 is only stable for about 1 week. In Na—PBS/Tween 20, MAG1-5 is stable for 6 weeks at ambient conditions.

While the present method of the invention is exemplified by purification of recombinantly-produced MAG from transformed host cells, the method is also amenable to purification of MAG naturally occurring within a cell and can be used to purify proteins from solution, cell homogenates, cell culture supernatants, or isolated cellular sub-fractions. While the present invention has been described in terms of specific methods and compositions, it is understood that variations and modifications will occur to those skilled in the art upon consideration of the present invention.

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

1. A method of purifying recombinant extracellular domain myelin associated glycoprotein (MAG) constructs comprising the steps of:
transfecting cells with a vector having a nucleic acid sequence encoding an affinity-tagged MAG construct and capable of expressing the affinity-tagged MAG construct comprising at least one Ig domain;
culturing the transfected cells in a medium such that the cells express the affinity-tagged MAG construct;
contacting a MAG construct-containing conditioned medium with a metal ion affinity chromatography resin, charged with a divalent metal ion; and
eluting a purified affinity-tagged MAG construct.

2. The method of claim 1, wherein the step of transfecting cells further comprises transfecting Chinese Hamster Ovary (CHO) cells.

3. The method of claim 1, wherein the method further comprises stably transfecting cells.

4. The method of claim 1, wherein the method further comprises selecting a resin having at least one divalent metal ion selected from the group consisting of nickel, cobalt, copper, cadmium, calcium, iron, zinc, and strontium.

5. The method of claim 4, wherein the divalent metal ion is nickel.

6. The method of claim 4, wherein the divalent metal ion is cobalt.

7. The method of claim 1, wherein the method further comprises selecting the resin from the group consisting of nickel-nitrilotriacetic acid resin and TALON™ resin.

8. The method of claim 1, wherein the method further comprises expressing affinity-tagged MAG with a polyhistidine tail.

9. The method of claim 1, wherein the method further comprises expressing affinity-tagged MAG with a FLAG tag with an amino acid sequence DYKDDDDK.

10. The method of claim 1, wherein the step of culturing the transfected cells further comprises expressing affinity-tagged MAG comprising at least two Ig domains.

11. The method of claim 1, wherein the step of culturing the transfected cells further comprises expressing affinity-tagged MAG comprising at least three Ig domains.

12. The method of claim 1, wherein the step of culturing the transfected cells further comprises expressing affinity-tagged MAG comprising at least four Ig domains.

13. The method of claim 1, wherein the step of culturing the transfected cells further comprises expressing glycosylated MAG.

14. The method of claim 1, wherein the step of culturing transfected cells further comprises culturing the cells to confluency in a medium comprising FBS and then changing to a serum-free medium.

15. The method of claim 1, wherein the step of culturing transfected cells further comprises culturing the cells in a medium comprising methotrexate.

16. The method of claim 1, wherein the step of eluting the purified affinity-tagged MAG further comprises at least one of a change pH, a chelating agent and a competitive ligand.

17. The method of claim 16, wherein the chelating agent is ethylenediamine tetraacetic acid in an eluting solution having a pH greater than about 7.

18. The method of claim 16, wherein the competitive ligand is imidazole.

19. The method of claim 1, wherein the method further comprises storing the purified affinity-tagged MAG in a buffer comprising Na₂HPO₄, NaCl, and a pH greater than about 7.0.

20. The method of claim 19, wherein the method further comprises storing the purified affinity-tagged MAG in a buffer comprising imidazole.

21. The method of claim 19, wherein the method further comprises storing the purified affinity-tagged MAG in a buffer comprising a detergent.

22. The method of claim 19, wherein the method further comprises storing the purified affinity-tagged MAG in a buffer comprising about 0.1% Tween 20.

23. A purified, glycosylated human recombinant extracellular domain myelin associated glycoprotein (MAG) construct prepared by a process comprising the steps of:
culturing transfected cells such that the cells express an affinity-tagged MAG construct;
contacting a MAG construct-containing conditioned media with a metal ion affinity chromatography resin, charged with a divalent metal ion; and
eluting a purified affinity-tagged MAG construct, wherein the eluted purified affinity-tagged MAG construct is greater than 90% pure.

24. The purified, glycosylated human recombinant extracellular domain myelin associated glycoprotein (MAG) construct of claim 23, wherein the transfected cells are transfected Chinese Hamster Ovary (CHO) cells.

25. The purified, glycosylated human recombinant extracellular domain myelin associated glycoprotein (MAG) construct of claim 23, wherein the step of eluting a purified affinity-tagged MAG construct further comprises eluting a purified affinity-tagged MAG construct that is greater than 95% pure.

26. The purified, glycosylated human recombinant extracellular domain myelin associated glycoprotein (MAG) construct of claim 23, wherein the MAG construct comprises an amino acid sequence that is substantially homologous with the amino acid sequence depicted in SEQ ID NO:2.

27. The purified, glycosylated human recombinant extracellular domain myelin associated glycoprotein (MAG) construct of claim 23, wherein the MAG construct comprises an amino acid sequence substantially homologous with the amino acid sequence depicted in SEQ ID NO:3.

28. The purified, glycosylated human recombinant extracellular domain myelin associated glycoprotein (MAG) construct of claim 23, wherein the MAG construct comprises at least two N-linked glycosylation sites.

29. The purified, glycosylated human recombinant extracellular domain myelin associated glycoprotein (MAG) construct of claim 23, wherein the MAG construct comprises at least 5% by weight carbohydrate.

30. A method for producing an extracellular domain myelin associated glycoprotein (MAG) comprising:
contacting a MAG-containing media with an immobilized metal affinity chromatography (IMAC) resin charged with a divalent metal ion;
washing the IMAC resin with at least one IMAC wash solution; and
eluting the IMAC resin with an eluting solution to obtain a purified MAG solution.

31. The method of claim 30, wherein the method further comprises selecting a divalent metal ion from the group consisting of nickel, cobalt, copper, iron, calcium and zinc.

32. The method of claim 31, wherein the divalent metal ion is nickel.

33. The method of claim 31, wherein the divalent metal ion is cobalt.

34. The method of claim 30, wherein the method further comprises the step of culturing transfected cells to confluence in medium comprising about 10% FBS and about 100 nM methotrexate, such that the cells express a MAG construct comprising at least one Ig domain.

35. The method of claim 30, wherein the purified MAG solution comprises affinity tagged MAG.

36. The method of claim 30, wherein the purified MAG solution comprises MAG with a polyhistidine tail.

37. The method of claim 30, wherein the purified MAG solution comprises MAG with a FLAG tag.
